(19) United States
(12) Patent Application Publication
(10) Pub. No.: US 2011/0166622 A1
(43) Pub. Date: Jul. 7, 2011

(54) SPHERICAL VIBRATING PROBE APPARATUS AND METHOD FOR CONDUCTING EFFICACY ANALYSIS OF PAIN TREATMENT USING PROBE APPARATUS

(75) Inventors: John Crosson, Beverly Hills, CA (US); David Weinkle, Cypress, CA (US)

(73) Assignee: NewLife Sciences LLC, Merrimack, NH (US)

(21) Appl. No.: 13/047,990

(22) Filed: Mar. 15, 2011

Related U.S. Application Data
(63) Continuation of application No. PCT/US2009/056990, filed on Sep. 15, 2009.
(60) Provisional application No. 61/097,037, filed on Sep. 15, 2008.
(60) Provisional application No. 61/097,054, filed on Sep. 15, 2008.

(51) Int. Cl. A61N 1/00

(52) U.S. Cl. 607/46

(57) ABSTRACT

A patient treatment unit and method analyzes and treats pain in tissues by applying an electrical pulse train and a galvanically isolated stimulus voltage to affected tissues using vibrating spherical tip probes. A range of probe diameters is used to provide a range of applied current densities. The impedance of the affected tissue is measured, tracked, and correlated to a level of pain while treatment is in progress. Impedance is used as real-time feedback, and current and voltage applications are adjusted accordingly. A patient treatment unit includes a probe stimulus generator connected to the spherically tipped probes. The unit further includes an impedance analysis circuit that senses voltage and current via the probes when they are contacting the patient. A monitor is electrically coupled to the body impedance analysis circuit and provides an indication of the measured impedance indicative of the patient's level of pain.

### CROSS-REFERENCE TO RELATED APPLICATION

This application is a continuation of prior International Patent Application Serial No. PCT/US2009/056990, filed Sep. 15, 2009, entitled “Spherical Vibrating Probe Apparatus and Method for Conducting Efficacy Analysis of Pain Treatment Using Probe Apparatus,” which claims the benefit of U.S. Provisional Patent Application Ser. No. 61/097,037, entitled “Efficacy Analysis Concurrent with Treatment” filed on Sep. 15, 2008 and U.S. Provisional Patent Application Ser. No. 61/097,054, entitled “Spherical/Vibrating Probe Tips” filed on Sep. 15, 2008, all of the above applications are incorporated herein by reference in their entirety.

### COPYRIGHT

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### FIELD OF INVENTION

The present disclosure relates to a patient treatment unit and method for analyzing and treating pain in human or animal tissues. More particularly, this disclosure relates to a spherical vibrating probe apparatus and methods for treating pain with the probe apparatus and conducting efficacy analysis of the pain treatment during treatment.

### BACKGROUND OF THE INVENTION

Electrical stimulation may be used for pain management. One such therapy is transcutaneous electrical nerve stimulation (TENS) therapy, which provides short-term pain relief. Electrical nerve stimulation and electrothermal therapy may also be used to relieve pain associated with various conditions, including back pain. Additionally, intradiscal electrothermal therapy (IDET) is a treatment option for patients with low back pain resulting from intervertebral disc problems.

Pain is typically attributable to a stimulus on nerve endings, which transmits signal impulses to the brain. This type of pain is referred to as nociceptive pain, a somatic sensation of pain, where a patient is made aware of potential tissue damage by neural processes encoding and processing noxious stimuli. The sensation is initiated by nociceptors that detect mechanical, thermal, or chemical changes above a pain threshold. Once stimulated, a nociceptor transmits a signal within the central nervous system through neurons. Each neuron transmits impulse information about the stimulus on the nerve endings along portions of the central nervous system transmission pathway.

Non-nociceptive pain is referred to as neuropathic pain or neuralgia. Neuralgia is pain produced by a change in neurological structure or function. Unlike nociceptive pain, neuralgia exists with no continuous nociceptive input. That is, neuralgia may develop without any actual impending tissue damage. Neuralgia may involve a disease of the nervous system, including an underlying disease process or injury, or from inflammation, infection, and compression or physical irritation of a nerve. Neuralgia is a form of chronic pain and can be extremely difficult to diagnose and treat.

Pain sensations may be gated naturally, such as when pain sensation is inhibited by activation of large diameter afferent neurons activated by vibration, such as when someone burns their hand, and it is involuntarily shaken in response. Transcutaneous electrical nerve stimulation also employs this technique by applying electrical nerve stimulating impulses from an external stimulator to reduce transmission of pain signals to the brain.

Transcutaneous electrical nerve stimulation (TENS) therapy may be used to treat both nociceptor pain and neuralgia. In TENS therapy, an electrical current is applied through the skin near the source of pain. The current is often delivered via electrodes. The current from the electrodes stimulates nerves in the affected area and sends signals to the brain that activate receptors in the central nervous system to reduce normal pain perception.

In a “Textbook of Pain” (Butler & Tanner Ltd., 3^{rd} Ed. 1994, pp. 59-62), authors Melzack and Walls proposed a gate theory to describe the manner in which transcutaneous electrical nerve stimulation devices interfere with pain. Melzack and Walls suggest that TENS devices generate an artificial abnormal noise on the neural pathways that are shared with the pain fibers conducting the real pain impulses. When the transmission of pain impulses from that region of the body are received by the central nervous system, the impulses are “gated.” That is, the transmission of the pain impulses is altered, changed, or modulated in the central nervous system by the artificial signals. As the central nervous system receives the barrage of signals from the stimulated region of the body, a neurological circuit closes a gate and stops relaying the pain impulses to the brain.

Gating is affected by the degree of activity in the large diameter and the small diameter nerve fibers. Nerve transmissions carried by large nerve fibers travel more quickly than nerve transmissions carried by small nerve fibers. As such, transcutaneous electrical nerve stimulation to large nerve fibers travels to the brain more quickly and are more powerful than pain impulses carried by smaller nerve fibers. Thus, the transcutaneous electrical impulses often arrive at the brain sooner than the pain nerve impulses, and the sensation of the large nerves overrides and blocks out the sensations from the smaller pain nerves. That is, impulses along the larger fibers tend to block pain transmission (close the gates) and more activity in the smaller fibers tends to facilitate transmission (open the gates). The gating mechanism in the spinal cord is affected by descending impulses from the brain. Large fibers may activate specific cognitive processes in the brain, which then influence the gate by downward (descending) impulse transmission.

Another theory regarding the pain reducing effect of transcutaneous electrical nerve stimulation devices is based on the understanding of serotonin and other chemical neurotransmitters that participate in the pain and the pain reduction processes in the central nervous system. Transcutaneous electrical nerve stimulation devices produce their effects by activating opioid receptors in the central nervous system. For example, high frequency transcutaneous electrical nerve stimulation activates delta-opioid receptors both in the spinal cord and supraspinally in the medulla, while low frequency transcutaneous electrical nerve stimulation activates mu-opioid receptors both in the spinal cord and supraspinally. Further high frequency transcutaneous electrical nerve stimulation reduces excitation of central neurons that transmit nociceptive information, reduces release of excitatory neurotransmitters such as glutamate, and increases the release of inhibitory neurotransmitters, including GABA, in the spinal cord, and activates muscarinic receptors centrally to produce analgesia. Low frequency TENS also releases serotonin and activates serotonin receptors in the spinal cord, releases GABA, and activates muscarinic receptors to reduce excitability of nociceptive neurons in the spinal cord.

By applying an electrical field to nervous system tissue, electrical stimulation can effectively reduce or mask certain types of pain transmitted from regions of the body. Pain perception may be inhibited by the applied electrical signals interfering with nerve transmission pathways carrying a pain transmission.

However, electrical stimulation intended to manage or control a pain condition may inadvertently interfere with other nerve transmission pathways in adjacent nervous tissue. Because neurostimulation devices must apply electrical energy across a wide variety of tissues and fluids, the amount of stimulation energy needed to provide the desired amount of pain relief is difficult to precisely control. As such, increasing amounts of energy may be required to ensure sufficient stimulation energy reaches the desired stimulation area. However, as the applied stimulation energy increases, so does the likelihood of damage of surrounding tissue, structures, or neural pathways.

In order to provide pain relief, the targeted tissue must be stimulated, but the applied electrical energy should be properly controlled, and the amount and duration of energy applied to surrounding or otherwise non-targeted tissue must be minimized or eliminated. An improperly controlled electric pulse may not only be ineffective in controlling or managing pain, but it may inadvertently interfere with the proper neural pathways of adjacent spinal nervous tissue.

### SUMMARY OF THE INVENTION

A system and method in accordance with the present disclosure uses a spherical vibrating probe tip to deliver stimulation energy to a patient precisely and accurately and avoids many of the pitfalls of conventional systems. A system and method of the present disclosure conducts an efficacy analysis concurrent with the spherical vibrating probe treatment to determine the degree to which the treatment is effective.

Impedance of body tissue changes with increasing electrical potential. That means that if the impedance measuring device uses a higher measuring voltage, a lower impedance will be measured. Measuring at actual treatment potential gives an important measure of change in body impedance. Conventional measurements of body impedance are not as accurate an indicator of the impedance that the delivered stimulation energy pulses are experiencing. In some cases, the potential used for treatment may create great discomfort if allowed to remain applied to the skin for a duration required to take a normal impedance measurement. The development of special circuitry would be required to measure in situ impedance.

A system and method in accordance with the present disclosure measures actual in body impedance while the transcutaneous electrical nerve stimulation treatment is in progress, thereby negated the need for stopping treatment to enter an analysis mode. In transcutaneous electrical nerve stimulation treatments, as well as in other electrical stimulation protocols, there may be a need to measure whether there has been any benefit from the treatment. In conventional transcutaneous electrical nerve stimulation treatment, the method of guiding the treatment is to pause in the application of the stimulation treatment and to take an impedance measurement of the body. If the body impedance has been lowered, the treatment is deemed to be successful. If there has been no reduction in body (tissue) impedance, then the treatment continues in that anatomical location of treatment until a drop in impedance is detected during a measurement pause.

A system and method in accordance with the present disclosure provides a spherical vibrating probe tip, with which electrical stimulation is provided to a patient. A means of measuring body impedance is provided during treatment, without pausing treatment. This provides immediate feedback to the healthcare professional administering treatment and eliminates the application of non-treatment currents, thereby providing an additional measure of patient safety. The patient is not exposed to non-treatment currents that might find a ground path through the patient.

A system and method of the present disclosure employs a spherical vibrating or non-vibrating probe tip to introduce electrical stimulation to the body. The spherical tipped probes provide a concentrated entry point for the current established between the probes. This entry point provides a greater current density (in mA/cm²) than possible with conventional conductive treatment pads, which typically range from 1 square inch to 8 square inches. The large area of the pads results in a current that is very diffuse, and may not be able to penetrate the dermis and therefore may be below the therapeutic value required. The spherical vibrating tips can be from ⅜″ in diameter to 1/16″ in diameter, thereby providing an effective current range due to a broad surface area of the voltage application. The spherical vibrating tip can be from 2 to 3 inches long and screw into the probe handle.

Additionally, the spherically-tipped probes provide the ability to modify the point of current entry in the affected tissue, thereby stimulating tissue from different directions and axes. The direction and angle at which the probe applies current to the affected tissue can be modified by the health care provide during treatment as the efficacy measurements are evaluated.

The probe can vibrate to provide further stimulation to the affected tissue. The axis of vibration can be axial, transversal, or radial, and can be driven at frequencies determined to be most beneficial for therapy. The vibrational motion can be imparted by electromagnetism as in a solenoid, or by piezo-electric actuators if relatively higher frequencies are desired. The probe vibrating frequency can also be variable, to tune for different patients, body tissues, and maladies. A patient's pain sensation may be inhibited by activation of large diameter afferent neurons activated by a spherically-tipped vibrating probe in accordance with aspects of the present disclosure. Beneficial effects of treatment using a system and method in accordance with aspects of the present disclosure can come from this applied vibrational stimulation due to enhanced blood and intercellular tissue fluid flow.

Further, pain relief from TENS may decline by 40% for many patients over the period of a year. There is evidence that some patients habituate to TENS currents owing to a progressive failure of the nervous system to respond to monotonous stimuli. Studies suggest that the nervous system may filter out monotonous system responses associated with TENS. The vibrating spherical probes disclosed herein further address this habituation response.

A patient treatment unit and method in accordance with aspects of the present disclosure analyzes and treats pain in human or animal tissues by applying an electrical pulse train to the affected tissue. The impedance of the affected tissue is measured, and the measured impedance is correlated to a level of pain in the patient. While monitoring the impedance, an additional pulse train is further applied and manipulated based upon the monitored impedance to reduce the patient's pain. The patient treatment unit includes a probe stimulus generator that outputs an electrical pulse train sequence or other specific electrical waveforms. The probe stimulus generator controls the pulse frequency and the pulse width of the electrical pulse train. The pulse width and carrier current can be varied to control the intensity of the electrical pulse train. The electrical pulse sequence output by the probe stimulus generator can include a stimulus profile that is based upon the surface and tissue impedance of the patient or can be a modified electrical pulse train based on the impedance response of the tissue of the patient. Of course, other waveforms may also be used depending upon the desired frequency, pulse width, carrier current, waveform polarity, and intensity.

The patient treatment unit further includes a body impedance analysis circuit that senses voltage and current via the probes when the probes are contacting the patient. The sensed voltage and current provides a means to measure the impedance of the examined tissue as the treatment is performed and to vary the position of the applied electrical pulse train, the frequency of the pulse train, the pulse width, the carrier current, and the like, as treatment progresses and the applied waveforms reduce the patient's pain. The treatment unit also includes a monitor that is electrically coupled to the body impedance analysis circuit that provides an audio, visual, or other concurrent indication of the impedance, the sensed voltage, or the sensed current indicative of the patient's level of pain. In this fashion, the body impedance analysis circuit can be used to measure surface and tissue impedance of the patient as the treatment progresses using the sensed voltage or current from the probes. The body impedance analysis circuit can also be used to measure the electrical phase of the voltage and current sensed from the probes and can include a filtering circuit in which waveform ripples in the sensed voltage or current are corrected. The monitor device can include an audio output with a frequency cut off volume that provides an indication of the sensed voltage or current or a visual indication of the determined impedance.

The body impedance analysis circuit of the present disclosure accurately and effectively measures voltage, current, and impedance using the probes during the treatment. The body impedance analysis circuit employs electrical components with strict material tolerances that provide accurate impedance measurements over a wide range of patients, thereby enabling treatment planning and pain treatment of patients with many body types and impedances. The body impedance analysis circuit, in concert with a stable electrical pulse train provided by the probe stimulus generator that supplies stable waveforms with non-varying pulse amplitudes, pulse widths, and pulse frequencies, enable accurate impedance measurements that are less susceptible to electrical noise and frequency drift. The electrical pulse width, amplitude, and frequency can be controlled by a physician or other trained operator. As a result, the pain treatments can be carried out safely and effectively. Conventional transcutaneous electrical nerve stimulation devices were often susceptible to electrical noise and drift, which made it difficult for a physician or other care giver to properly determine the length and effectiveness of the treatment. Accurate waveform transmission and simultaneous impedance measurements provided by a system and method of the present disclosure enable safe and effective treatment.

Additionally, a patient treatment unit in accordance with aspects of the present disclosure can include a treatment counter circuit that detects and tracks an elapsed treatment time indicative of the time the primary probe is receiving the sequence of electrical pulses. The treatment counter circuit can be used to measure and track treatments for regulatory and insurance compliance and to ensure treatment efficacy and patient safety. Likewise, the patient treatment unit can also include a coil sense circuit that evaluates the presence of a probe connection and enables the probe stimulus generator when the probes are connected to the body impedance analysis circuit. The coils sense circuit can ensure that no electrical pulse train is generated when the probes are not properly connected.

The patient treatment unit in accordance with aspects of the present disclosure can further include a wall wart power supply circuit that provides a stable and regulated 12 volt DC power source to the patient treatment unit. The probe stimulus generator can provide a handshaking signal to the power supply circuit to check for a stable and regulated 12 volt DC power source. If the power supply voltages or currents are outside a specified acceptable range, the probe stimulus generator will not be enabled, and no treatment may commence. A visual or other indication of the status of the power source can be displayed using LEDs on the patient treatment unit or by an audio or other indication.

The treatment unit also includes a response level circuit that is used to measure and indicate the conductivity between the probes. Also, the patient treatment unit can further include an intensity adjustment circuit that is used to measure, indicate, and adjust the intensity of the electrical pulses. The intensity of the electrical pulses can be adjusted by adjusting a carrier current or the pulse width or the amplitude of the electrical pulse train. The patient treatment unit can also include a programming and debugging circuit that is used to configure the patient treatment unit and to debug processing errors in the patient treatment unit.

The patient treatment unit in accordance with aspects of the present disclosure uses electrical pulse trains to reduce chronic intractable pain. The treatment unit can also be used as an adjunctive treatment in the management of post-surgical and post-traumatic acute pain. The patient treatment unit in accordance with the present disclosure is a symptomatic treatment device, and as such, suppresses the sensation of pain that may otherwise signal potential tissue damage. The present disclosure includes a spherical vibrating probe apparatus to deliver stimulation energy to a patient while conducting an efficacy analysis concurrently with the treatment to determine the degree to which the treatment is effective.

These and other advantages, aspects, and features of the present disclosure will become more apparent from the following detailed description of embodiments and implementations of the present disclosure when viewed in conjunction with the accompanying drawings. The present disclosure is also capable of other embodiments and different embodiments, and details can be modified in various respects without departing from the spirit and scope of the present disclosure. Accordingly, the drawings and descriptions below are to be regarded as illustrative in nature, and not as restrictive.

### BRIEF DESCRIPTION OF THE DRAWINGS

The accompanying drawings illustrate an embodiment of the disclosure and depict the above-mentioned and other features of this disclosure and the manner of attaining them. In the drawings:

FIG. 1 is a functional block diagram illustrating a patient treatment unit in accordance with aspects of the present disclosure.

FIG. 2 is a top view illustration of a patient treatment unit in accordance with aspects of the present disclosure.

FIGS. 3A-3D illustrate a spherical vibrating treatment probe in accordance with aspects of the present disclosure.

FIG. 4A is an illustration of the initial device settings of a patient treatment unit in accordance with aspects of the present disclosure.

FIG. 4B is an illustration of the treatment time code of a patient treatment unit in accordance with aspects of the present disclosure.

FIGS. 5A-5D show impedance, power and frequency relationships for a patient treatment unit in accordance with aspects of the present disclosure.

FIGS. 6A-6B are process flow diagrams outlining a method of analyzing and treating pain using a patient treatment unit in accordance with aspects of the present disclosure.

FIG. 7 is a modified schematic diagram of a Probe Stimulus Generator illustrating the electrical noise-reduction and frequency drift reduction components incorporated into circuits in accordance with aspects of the present disclosure.

FIG. 8 is a functional block diagram illustrating an isolated data logger according to aspects of the present disclosure.

FIGS. 9A-9B illustrate a configuration of the spherical tipped vibrating probes in accordance with aspects of the present disclosure to measure the depth of penetration of treatment current.

### DETAILED DESCRIPTION OF THE INVENTION

The following detailed description of the disclosure refers to the accompanying drawings and to certain preferred embodiments, but the detailed description does not limit the disclosure. The scope of the disclosure is defined by the appended claims and equivalents as it will be apparent to those of skill in the art that various features, variations, and modifications can be included or excluded based upon the requirements of a particular use.

The patient treatment unit sends an electrical pulse train to the patient's tissues via vibrating spherically-tipped primary and secondary probes to provide nerve stimulation to relieve the patient's pain. The patient treatment unit in accordance aspects of the present disclosure receives impedance measurements from a patient's tissues using primary and secondary spherical probes as the treatment is in progress. As the electrical pulse train is applied, the impedance measurements are monitored. A drop in impedance is indicative of less resistance. The lower impedance measurements have been correlated to lower perceived levels of pain that patients experience. The patient treatment unit in accordance with aspects of the present disclosure receives impedance information from the patient's tissues, including the body's cellular network. By monitoring the received impedance information as the treatment is in progress, additional electrical pulse trains can be applied further until the efficacy of the treatment plateaus. The systems and methods of the present disclosure assess and treat pain experienced by the patient's tissues and other physical structures.

In assessing and treating pain, the systems and methods of the present disclosure employ spherically-tipped vibrating probes to apply electrical pulse trains at the site of pain, at the tissue abnormality, or upon selected nervous system trigger points or motor points. These trigger or motor points can also coincide with acupuncture or pressure points of the body. An electrical pulse train is transmitted into the tissue and encounters the inherent impedance signature produced by the tissue or subject matter under study. The impedance information is generated by this initial analysis and measurement and can be used as a baseline measurement to plan and evaluate treatment and to monitor the efficacy of the treatment as the treatment is in progress.

In addition to evaluating and characterizing a patient's degree of pain, the systems and methods of the present disclosure are also used to provide therapeutic action to alleviate the pain. The patient treatment unit and spherical vibrating probes can provide neural stimulation to alleviate pain, reduce healing time, and upon suitable repetition of therapy, result in long-term improved pain management of the afflicted area.

Pain is reduced or eliminated by means of the electrical pulse train effect on nociceptive afferent neurons, which are sensitive to electrical stimuli as well as noxious stimuli including thermal, mechanical, and chemical stimuli as described above.

An electrical device and method for analyzing and treating abnormality of human and animal tissues includes means for delivering an electrical pulse train having an output voltage in the approximate range of 50-60 volts and a peak pulse amplitude of 190 volts. The means for delivering the electrical pulse train provide a pulse rate range of 1-490 pulses per second and a pulse duration range of 0.24 to 0.74 milliseconds. Additionally, the means for delivering the electrical pulse train provide a maximum output current of 8.9 milliamps and a maximum charge per pulse of 7 microcoulombs. The electrical pulse train can include complex wave forms with variable frequency, variable pulse width, and AC-coupled rectangular pulses.

The system also includes means for detecting and measuring impedance of the patients' tissues during treatment and subjecting the tissues to an electrical pulse train. The system further includes means for generating and applying an electrical pulse train to the tissues to reduce or nullify pain impulse signals perceived by patients.

As outlined above, a patient treatment unit in accordance with the present disclosure analyzes and treats pain in human or animal tissues by applying an electrical pulse train to the affected tissue via a vibrating spherical probe. As the treatment progresses, the impedance of the affected tissue is measured, and the measured impedance is correlated to a level of pain in the patient. While monitoring the impedance, additional pulse trains can be further applied and manipulated based upon the measured impedance and the efficacy of the treatment can be assessed in an on-going fashion to reduce the patient's pain.

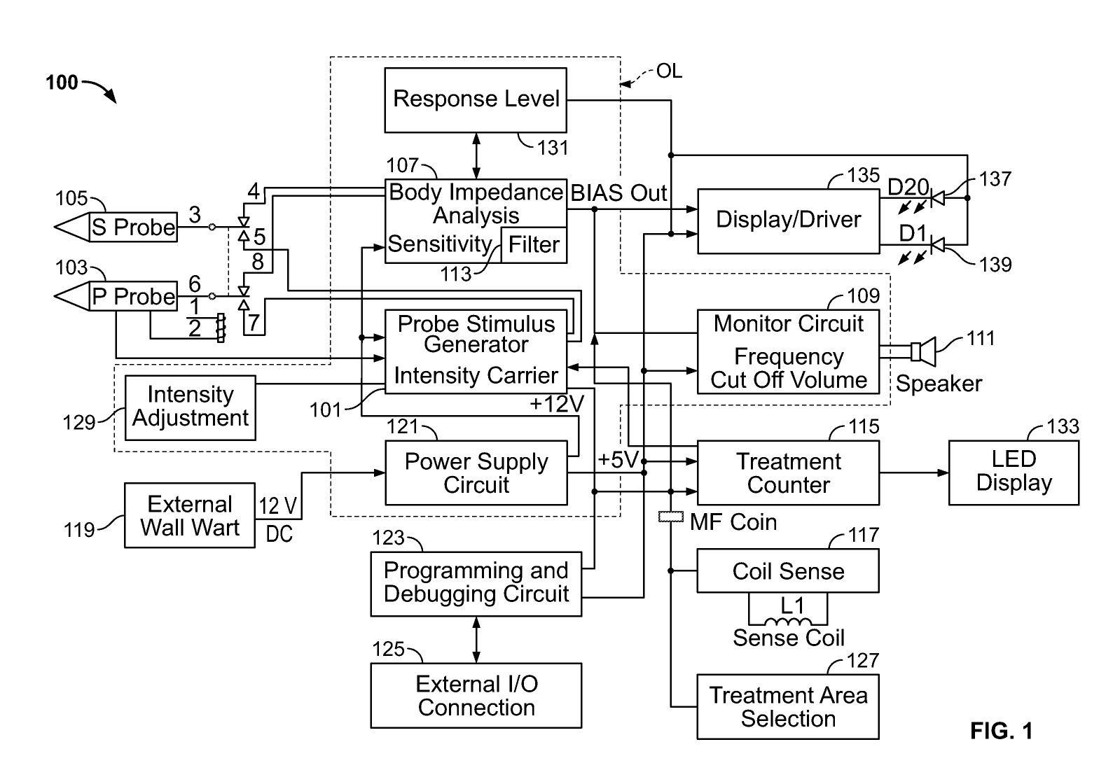

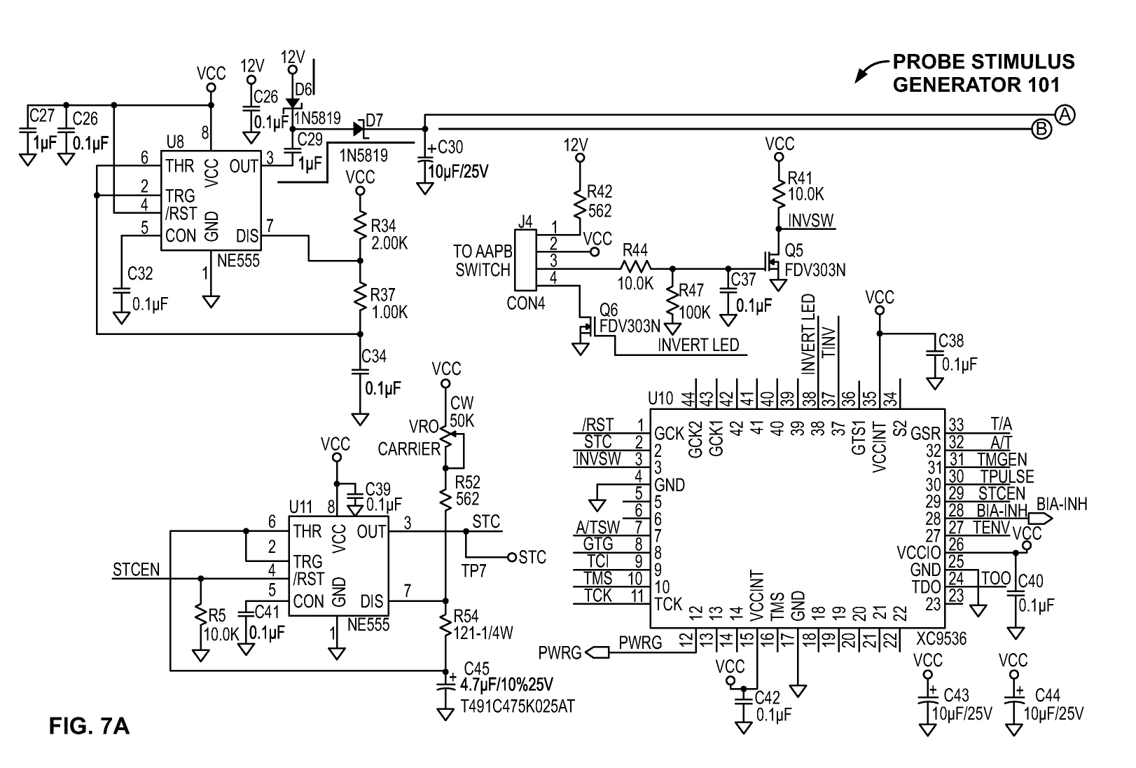

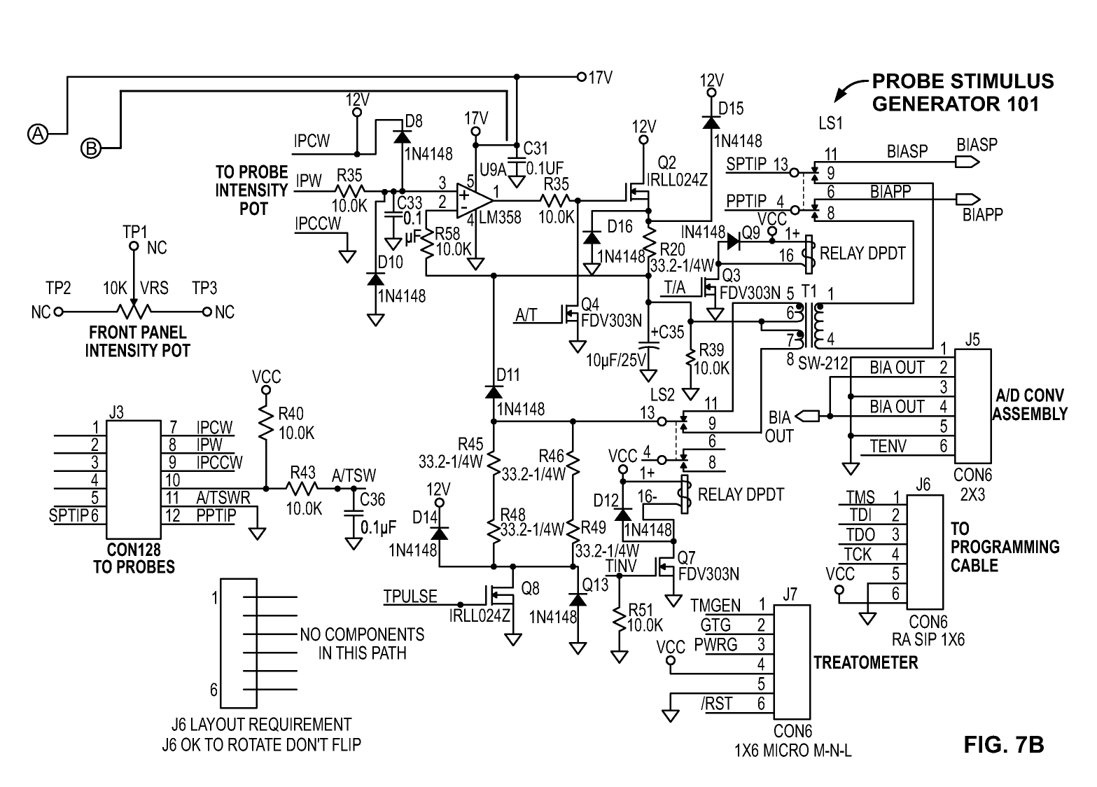

FIG. 1 shows a functional block diagram illustrating a patient treatment unit 100 in accordance with aspects of the present disclosure. The patient treatment unit 100 includes a probe stimulus generator 101 (also shown schematically in FIG. 7) that outputs an electrical pulse train sequence to the spherical vibrating probes 103, 105. The probe stimulus generator 101 controls the pulse frequency, the pulse width, and the polarity of the electrical pulse train. The pulse width and the carrier current can be varied to control the intensity of the electrical pulse train. Additionally, the probe stimulus generator outputs an electrical pulse train that is a clean waveform, largely free of electrical noise by using rigid electrical component tolerances in a carrier waveform generation circuit. For example, as shown in FIG. 7, the carrier waveform frequency is set using carrier adjustment VR6 in combination with capacitor C45. This RC circuit can be adjusted to produce the desired carrier frequency of the electrical pulse train. The RC circuit values provide a stable waveform, largely free of electrical noise. Similarly, once the carrier frequency is set, the waveform is not susceptible to frequency drift.

The probe stimulus generator 101 can use a number of different electrical pulse train configurations, depending upon the treatment at hand. For example, a number of different waveforms of variable amplitude can be selected. A basic square wave with a pulse width of 0.24 milliseconds and a pulse rate of 440 pulses per second with a pulse amplitude of 100 volts may be selected to treat lower back pain.

As shown by outline OL in FIG. 1, a number of the circuits 121, 107, 101, 121, 129, 109 can be physically mounted and manufactured on a single printed circuit board to reduce electrical noise between components and circuits. The printed circuit board can be a multi-layer printed circuit board to further reduce ambient electrical noise and to generate a clean and error-free pulse train.

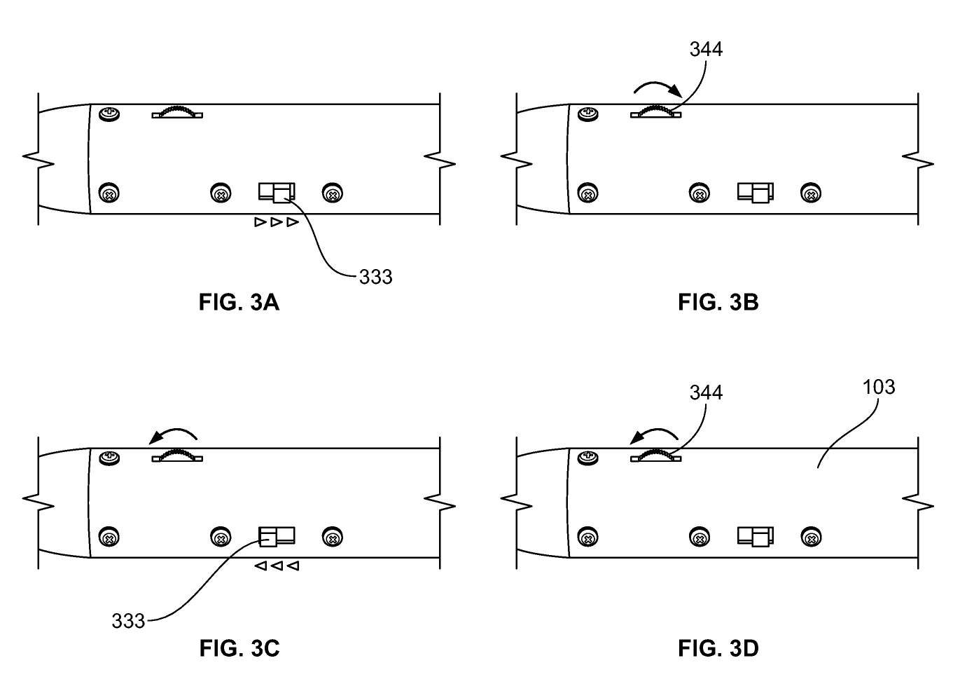

As also shown with regard to FIG. 7, probe stimulus generator 101 also includes internal monitor functions to ensure the safety and performance of patient treatment unit 100. For example, probe stimulus generator 101 monitors and checks power supply voltage from power supply circuit 121 as well as a coil sense indication from coils sense circuit 117 that the probes 103, 105 are properly connected across a proper tissue or patient. Further, treatment counter 115 provides a handshake signal indicating a ready condition that must be detected by probe stimulus generator 101 before a pulse train may be applied to a tissue. Probe stimulus generator 101 also includes level shifting circuitry that can be used to alter the carrier current as well as to shift the current and voltage limiting circuitry. Level shifting is provided by T1 where a variable 0-12 VDC is applied to pins 5 and 6 of T1, and the output of T1 is 12 times the applied voltage. This is a function of the turns ratio of the transformer T1. The variable voltage is under the control of the intensity dial 344 located on primary probe 103 (shown in FIG. 3). The probe stimulus generator 101 will not output the sequence of electrical pulses until the power supply handshake, the coils sense handshake, and the treatment counter handshake signals all indicate that these circuits 121, 117, 115 are in a ready condition.

The treatment unit also includes a pair of spherical vibrating probes 103, 105 for receiving the electrical pulse train and applying the pulse train to the patient's body. The pair of spherical vibrating probes includes primary spherical probe 103 and secondary spherical probe 105 that are both electrically coupled to the probe stimulus generator 101 to receive the sequence of electrical pulses.

A galvanically isolated stimulus voltage is applied across an anatomical area of the patient using vibrating spherical probes 103, 105. This isolated voltage safeguards the patient from electrical shock or electrocution as the treatment progresses. The applied voltage and current are measured in real time using vibrating spherical probes 103, 105, and the patient treatment unit 100 determines body impedance by dividing the applied voltage by the current of the measured stimulus voltage. This yields the body impedance in real time. The real-time impedance is monitored throughout the treatment process to determine the efficacy of the treatment.

Importantly, all the measurement and feedback of the measurement is done in an isolated manner so as to prevent any possibility of harm to the patient by having inadvertent currents pass through the patient's body. The only current passing through the patient's body should be supplied by the stimulation voltage and not the measurement circuitry.

Methods and systems for tracking improvement in pain reduction while treatment is in progress. In treatment involving electrical stimulation, a galvanically isolated stimulus voltage is applied across parts of the human body. This isolation is crucial to safeguard the patient from being electrocuted. The system measures the applied voltage and current in real time of the isolated stimulus voltage and allows the instrument to determine body impedance by dividing the applied voltage by the current of the measured stimulus voltage, yielding the body impedance in real time. All the measurement and feedback of the measurement is done in an isolated manner to prevent any possibility of harm to the patient by having inadvertent currents pass through the body. The only current passing through the body should be supplied by the stimulation voltage and not the measurement circuitry.

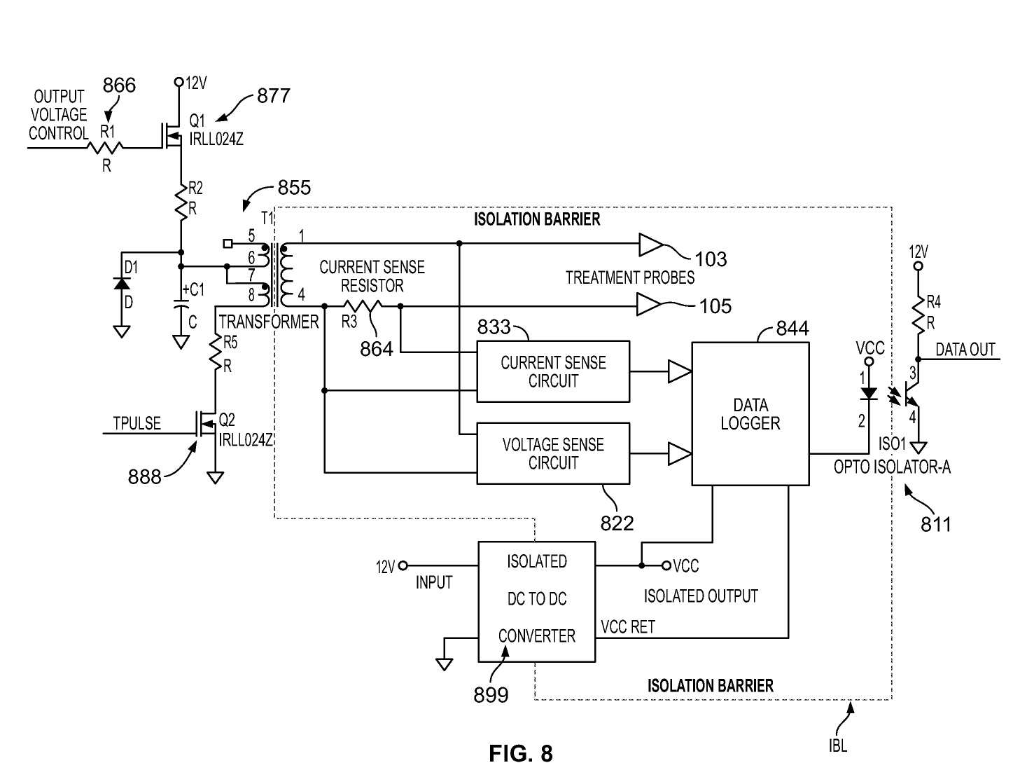

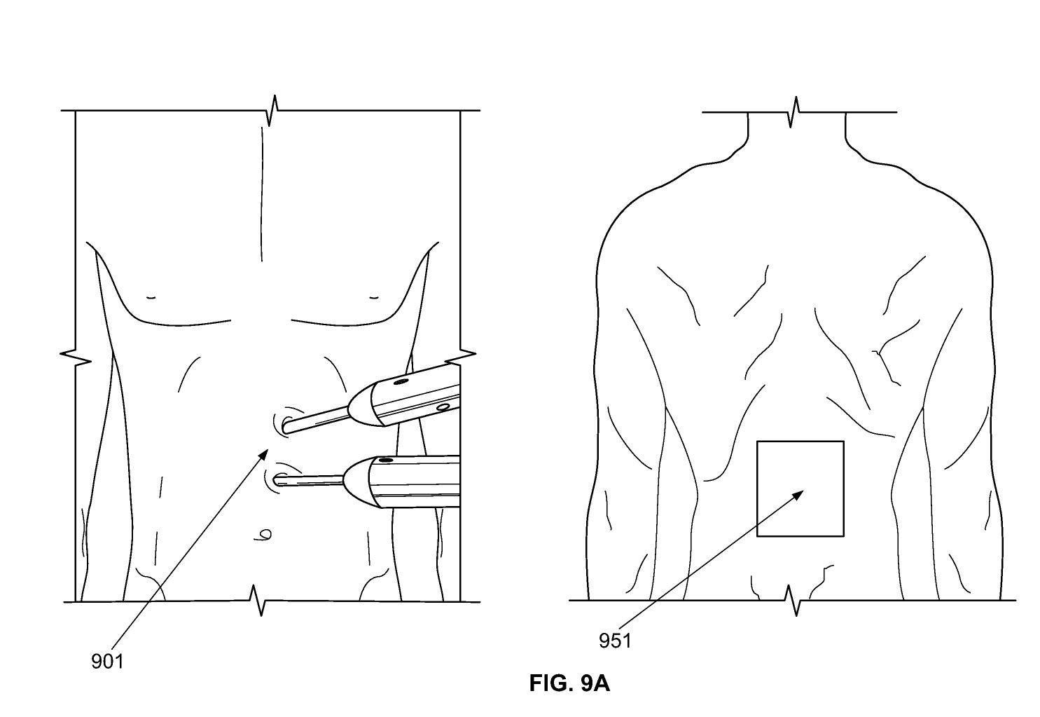

Primary vibrating spherical probe 103, as shown in FIGS. 3A-3D reads conductivity and impedance between primary vibrating spherical probe 103 and secondary vibrating spherical probe 105 in real-time as treatment is conducted. As further illustrated in FIG. 8, to measure impedance in real-time, a stimulation voltage is applied between the vibrating spherical probes 103, 105 to measure the impedance of the tissue to be examined. The stimulation voltage is isolated from the pulse generator circuit of the probe stimulus generator by transformer 855. As outlined above, any additional equipment or accessories that may be attached to the vibrating spherical probes or output circuit should be isolated from any circuit that could have a path to the AC mains, which includes the pulse generating circuit of the probe stimulus generator.

Voltage and current can be sensed and measured, and impedance readings are calculated. For example, the output pulse amplitude is controlled by the output voltage control input at resistor 866 into the gate of transistor 877. The pulse width and timing is controlled by TPULSE into transistor 888. Besides isolating the pulse, transformer 855 also serves to amplify the pulse from the 12 volt or less level to some much higher voltage determined by the turns ratio of transformer 855.

Anything within the isolation barrier line IBL is either galvanically or optically isolated so there will be no current passed over this barrier. Capacitive coupling may also be used to further isolate the current.

The isolated dc to dc converter 899 provides the isolated power to operate the data logger 844 electronics. The current sense circuit 833 senses the voltage across the low value resistor 864 to sense the current passing through the vibrating spherical probes 103, 105. The voltage sense circuit 822 senses the voltage across the vibrating spherical probes 103, 105. This sensed data is passed on to the data logger 844 circuit, which can either calculate the body impedance by dividing the sensed current into the sensed probe voltage, or pass on these parameters to other circuits where the calculation may be performed. As shown in FIG. 8, the data values are passed in this example as digitized data over an optically isolated serial bit stream through opto-isolator 811. The raw analog data can also be passed across this barrier by analog coupling schemes. This data can be presented to a user in a plotted format of impedance-versus-time to show the change in impedance value with treatment time for a particular probe placement.

As further shown in FIG. 3A, treatment switch 333 activates contact level display 214 to provide a visual indication of the conductivity and impedance of the tissue under examination. When pushed forward, treatment switch 333 activates treatment by completing a coil sense circuit 117 that enables probe stimulus generator 101 to generate an electrical pulse train output to treat the tissue under examination. When switched to treatment mode, the probe stimulus generator 101 receives a handshake signal from the treatment counter 115. In this fashion, probe stimulus generator 101 can provide output current to the vibrating spherical probes 103, 105 in the form of the electrical pulse train when the treatment counter 115 is in the circuit. The probe stimulus generator 101 also checks the power supply circuit 121 to ensure that proper power is provided prior to enabling output current in the form of an electrical pulse train. If the power is not adequate, or if the treatment counter 115 does not shake hands, the stimulus generator 101 is precluded from outputting the electrical pulse train. The various handshake checks are made by handshake controller U10 (best illustrated in FIG. 7). When the patient treatment unit 100 is in the treatment mode, the impedance between the probes (and therefore the impedance of the tissue under examination) is shown in contact level display 214. Display 214 shows a relative level of impedance. It establishes a nominal baseline value for a particular patient as an indicator to show a change in impedance of the affected tissue as treatment is ongoing. Additionally, primary vibrating spherical probe 103 includes an intensity dial 344 shown in FIG. 3B that controls the intensity of treatment. At the onset of treatment, the intensity dial 344 should be turned toward the back of the probe at its minimum setting. The intensity dial 344 is then turned forward toward the front of the probe 103 until the patient feels the carrier current, but is not uncomfortable.

The patient treatment unit 100 further includes a body impedance analysis circuit 107 that senses voltage and current via the vibrating spherical probes 103, 105 when the probes 103, 105 are contacting the patient. The sensed voltage and current provide a means to measure the impedance of the examined tissue in real-time and to vary the position of the applied electrical pulse train, the frequency of the pulse train, the pulse width, the carrier current, and the like, as the efficacy of the treatment is evaluated by monitoring the impedance. As indicated above with regard to the probe stimulus generator 101, by setting an accurate frequency, amplitude, and pulse width of the electrical pulse waveform, the body impedance analysis circuit is better able to determine an accurate impedance measurement of the examined tissue. Changes in impedance can then be measured causally based upon the applied treatment rather than ascribed to any drift in the carrier waveform frequency or electrical noise.

The treatment unit 100 also includes a monitor circuit 109 that is electrically coupled to the body impedance analysis circuit 107 and provides an audio, visual, or other indication of the impedance, the sensed voltage, or the sensed current indicative of the patient's level of pain. Body impedance analysis circuit 107 can also simultaneously sense voltage and current associated with skin and tissue measurements as well as convert sensed readings to characterize other properties of the measured tissue such as conductivity, impedance, and the like. The indication can be provided by speaker 111, a display monitor (not shown), or another indicator. In this fashion, the body impedance analysis circuit 107 can be used to measure surface and tissue impedance of the patient in real-time using the sensed voltage or current from the vibrating spherical probes 103, 105 and indicated to a physician or other operator. The body impedance analysis circuit 107 can also be used to measure the electrical phase of the voltage and current sensed from the vibrating spherical probes 103, 105 and can include a filtering circuit 113 in which waveform ripples in the sensed voltage or current are corrected. Display driver 135 can be used to illuminate LEDs 137, 139 to provide an indication of the sensitivity of the probe measurement. Of course, other visual or audio methods of indication can be used as well. The monitor circuit 109 can include an audio output to speaker 111 that includes a frequency cut off volume to provide an indication of the sensed voltage or current or a visual indication of the determined impedance.

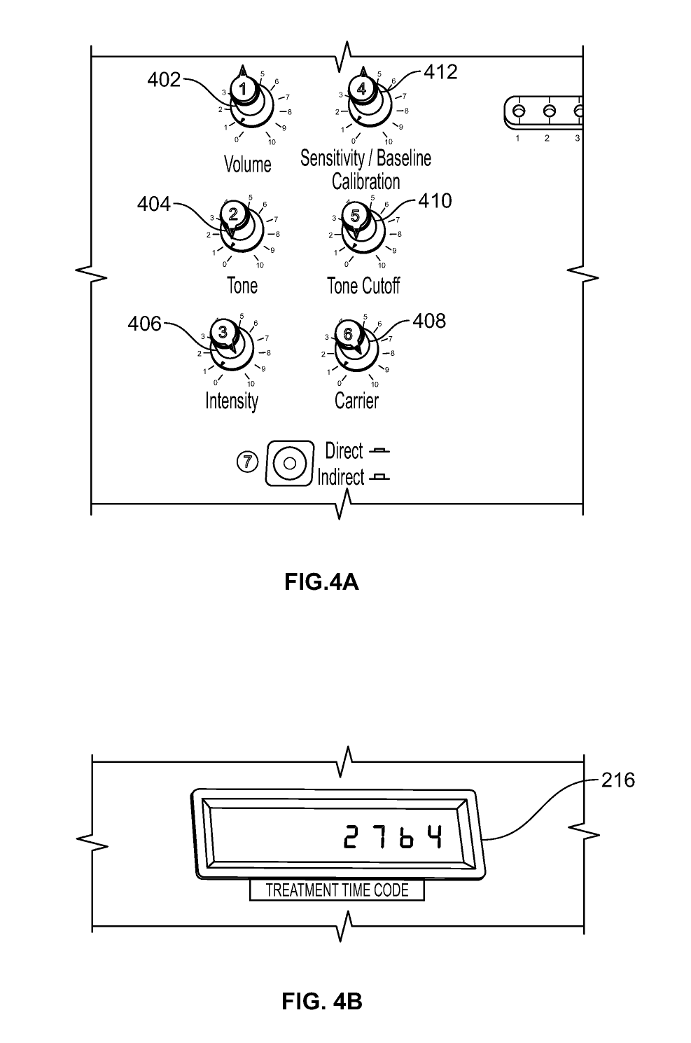

Additionally, the patient treatment unit 100 can include a treatment counter circuit 115 that detects and tracks an elapsed treatment time indicative of the time the primary vibrating spherical probe 103 is receiving the sequence of electrical pulses. The treatment counter circuit 115 can be used to measure and track treatments for regulatory and insurance compliance and to ensure patient safety. A visual indication of the treatment time can be presented using display 133, or a treatment time code as shown in FIG. 4A. Patient compliance with treatment is a medical concern regardless of the form of treatment. Patients must follow through with the prescribed treatments to ensure efficacy and to facilitate recovery. If a patient avoids treatment or takes part in the treatment in a manner not prescribed, the patient's noncompliance masks any effects of the treatment. This leads to great uncertainty as to the effectiveness of the prescribed therapy and whether the current level of treatment is appropriate, or if it is in need of adjustment or discontinuation. Patients are often unwilling to admit they are non-compliant, and when a treatment is difficult or painful, patients may choose to forgo or avoid the treatment despite proven therapeutic benefits. Misuse of the treatment weakens the economic and therapeutic incentives for health care providers and insurance companies to fund or cover the costs of the treatment.

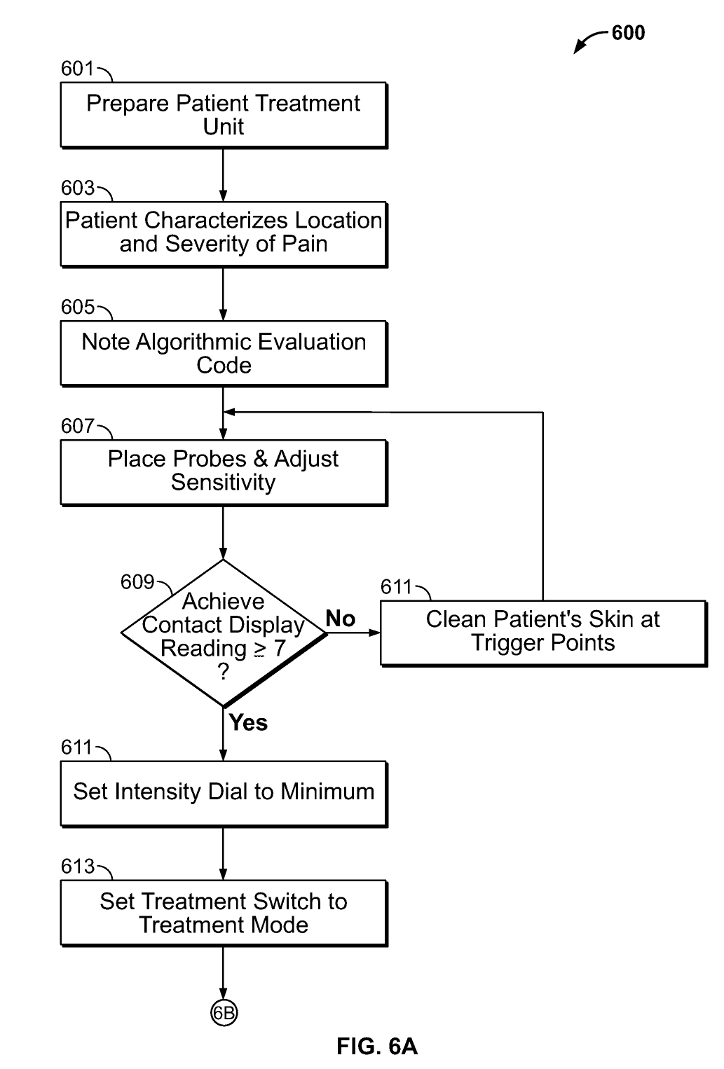

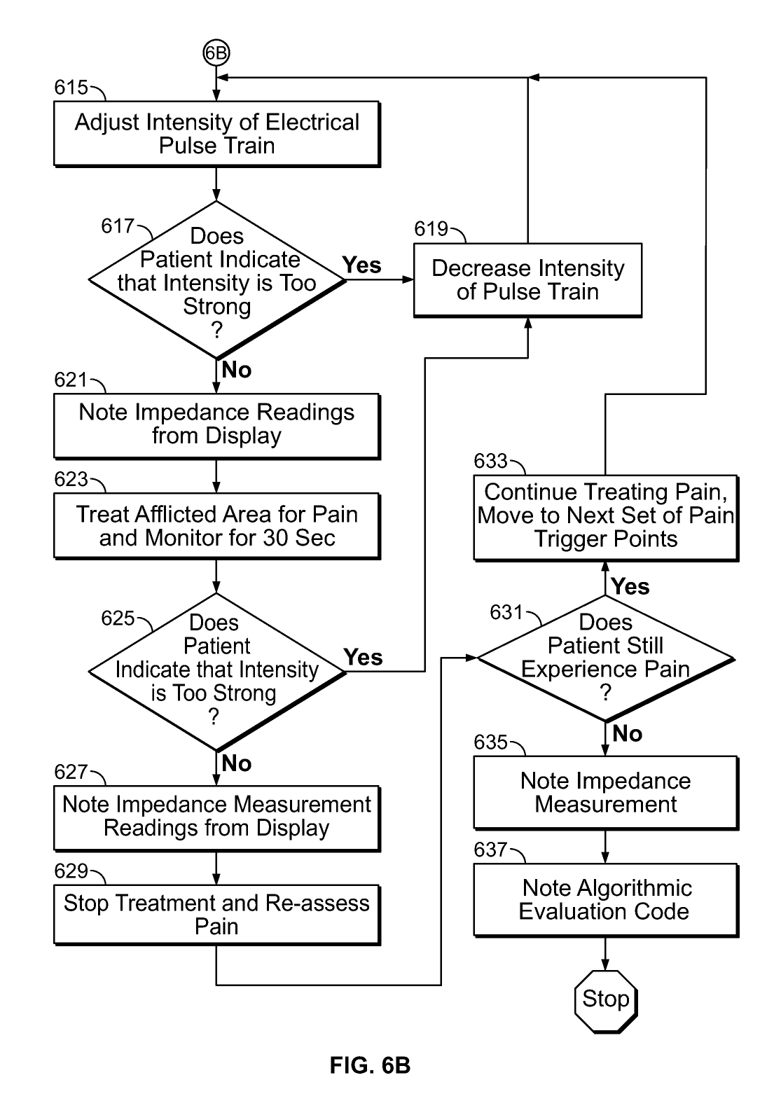

To ensure compliance for both medical outcomes and insurance requirements, the patient treatment unit 101 includes treatment time code display 216 as a compliance monitoring tool. Treatment time code display 216 tracks and displays the treatment time during which an electrical pulse train is applied to the affected patient tissues. The treatment time code (treatment) counter 115 runs continuously as long as the patient treatment unit 100 is in treatment mode and thereby tracks actual treatment time. Each time the patient treatment unit 100 is powered on, the current software revision will be illuminated in the treatment time code field display 216. The software version number remains illuminated until the patient treatment unit 100 is turned off or until the treatment is activated by pushing forward the treatment switch 333 on the primary vibrating spherical probe 103 as shown in FIGS. 3A-3D and described below with regard to FIGS. 6A-6B. Once the treatment switch 333 is activated and treatment begins, the treatment counter 115 will take over the treatment time code display 216, and the code display 216 will track the time elapsed via a hexadecimal display or other indicator. The treatment time code display 216 will continue to count as long as the primary vibrating spherical probe 103 remains in treatment mode. Once the treatment switch 333 is deactivated, the treatment time code 216 will stop incrementing but will remain visible. Treatment time code display 216 will not increment until the treatment switch 333 on primary vibrating spherical probe 103 is once again moved forward to re-start additional treatment. At that time, the treatment time code display 216 will again continue to increment. With each patient treatment session, the starting value for treatment time code display 216 must be noted upon commencement of the treatment session and at the end of the treatment session, the end value on the treatment time code display 216 must be noted. These values should be recorded in the patient's file to track treatment times and compliance.

Likewise, the patient treatment unit 100 may also include a coil sense circuit 117 that evaluates the presence of a probe connection and enables the probe stimulus generator 101 when the vibrating spherical probes 103, 105 are connected to the body impedance analysis circuit 107. The coils sense circuit 117 ensures that no electrical pulse train is generated when the probes 103, 105 are not properly connected.

The patient treatment unit 100 can further include a wall wart power supply 119 and a power supply circuit 121 that provides a stable and regulated 12 volt DC power source to the patient treatment unit 100. The stable and regulated power source helps provide an electrical pulse train free from ambient electrical noise. Further, the patient treatment unit employing a wall wart power supply 119 and power supply 121 of the present disclosure is less susceptible to fluctuations in AC input power typically provided by convenience outlets and other conventional power receptacles. The wall wart power supply 119 and power supply circuit 121 promote treatment efficacy and lower treatment costs by eliminating the need to replace batteries during treatment or at other inopportune times as may be the case with conventional systems.

Likewise, the wall wart power supply 119 and power supply circuit 121 of the present disclosure eliminates the need to monitor battery power and to make adjustments to power output once the overall power level of the battery source has dropped beneath a threshold power level. By employing the wall wart power supply 119 and power supply circuit 121 of the present disclosure, the output signal (electrical pulse train) of the patient treatment unit is less susceptible to fluctuations following power disruptions, defective operations, or operator misuse.

The patient treatment unit 100 can also include a programming and debugging circuit 123 that is used to configure the patient treatment unit 100 and to debug processing errors in the patient treatment unit 100. The programming and debugging circuit 123 can be integral hardware to patient treatment unit 100 or can be deployed via an external input/output connection 125 to accommodate a laptop computer or other device that can provide input commands and receive output commands to program, analyze, and process computer instructions used to carry out a method of the present disclosure using patient treatment unit 100. The programming and debugging circuit 123 can also be used to update the computer program instructions used to carry out a method of the present disclosure.

The treatment unit 100 also includes a response level circuit 131 that is used to measure and indicate the conductivity or impedance between the vibrating spherical probes 103, 105 in real-time. Also, the patient treatment unit 100 can further include an intensity adjustment circuit 129 that is used to measure, indicate, and adjust the intensity of the electrical pulses. The intensity of the electrical pulses can be varied by adjusting a carrier current or the pulse width of the electrical pulse train using the intensity dial 344 shown in FIG. 3D. As shown further in FIG. 7, the frequency of the pulses is changed by adjusting VR6. The pulse width can be modified by changing R54. The carrier current is adjusted by the intensity knob 344 on primary probe 103 (as shown in FIG. 3). Intensity knob 344 is also shown in FIG. 7 as the Probe Intensity Pot.

The electrical output specifications of patient treatment unit 100 are shown below in Table 1:

TABLE 1

| | |
|---|---|
| Power Supply | 115 VAC, 60 Hz |
| | 12 volt, DC output |
| Maximum Power Consumption | 21 W |
| Output voltage | Range of normal use: 50-60 V |
| | Peak pulse amplitude: 190 V |
| Pulse Rate | 1-490 Pulses/second, ±6% |
| Pulse Duration | 0.24-0.74 millisecond |
| Output Current (maximum) | 8.9 milliamps |
| Maximum charge per pulse | 7 micro coulombs |
| Wave Form | Complex pulse trains: variable |
| | frequency, variable pulse width, |
| | AC-coupled rectangular pulse |

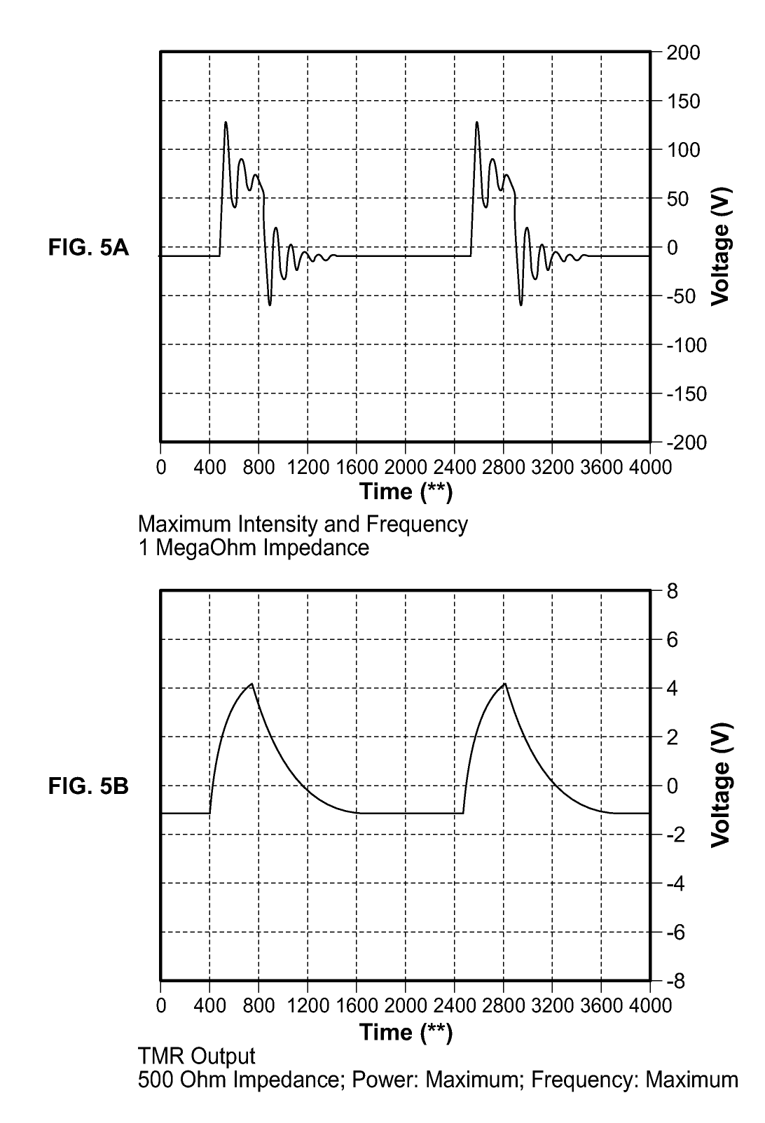

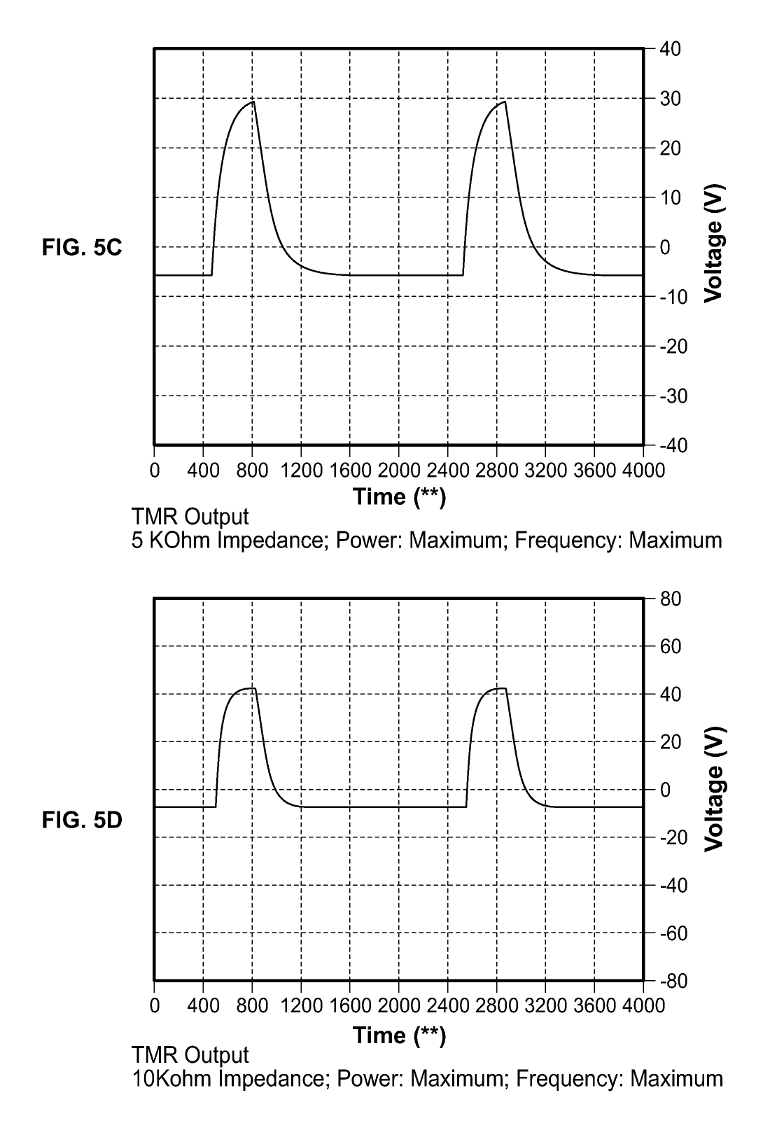

FIGS. 5A-5D show output waveforms of a patient treatment unit 100 in accordance with the present disclosure. FIGS. 5A-5D illustrate a number of impedance, power, and frequency relationships. For example, FIG. 5A shows a frequency response using a 1 MΩ maximum impedance. The output waveform varies depending on the load as shown in FIGS. 5B-5D. That is, FIG. 5B shows voltage versus time at 500 ohms. FIG. 5C shows voltage versus time at 5 kΩ ohms, and FIG. 5D shows voltage versus time at 10 kΩ. Changes in load affect both pulse duration and maximum pulse frequency. For example, maximum pulse rate frequency is in a range of 490 Hz±6% from 500 ohms to 1 MΩ. Additionally, the pulse frequency of the electrical pulses can be in a range of substantially 4 kHz to 20 kHz±6% with 500 ohms to 1 mega-ohm of resistance across the vibrating spherical probes. Lower impedances have lower maximum pulse rates, while pulse width is fixed for a given impedance. For example, pulse width is 0.74 milliseconds at 500 ohms and is 0.24 milliseconds at 1 MΩ.

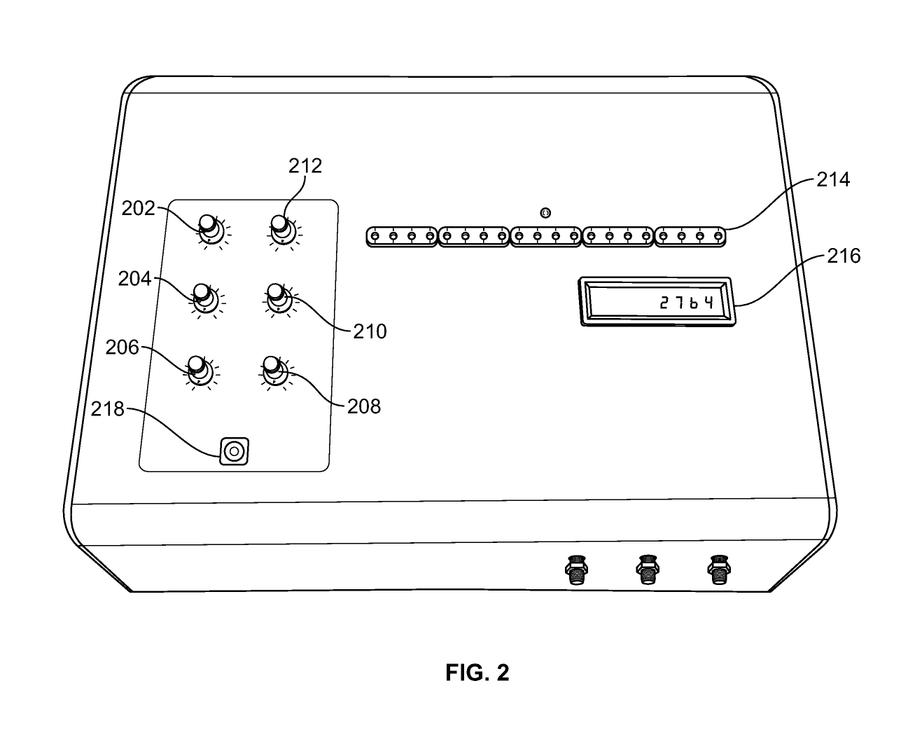

FIGS. 6A-6C illustrate a method 600 to control pain using a patient treatment unit 100 in accordance with the present disclosure. In block 601, a physician or other licensed operator makes the patient treatment unit 100 ready for use by preparing the initial device settings. As illustrated in FIG. 2 and in FIG. 4A, the initial device settings include the volume, tone, intensity, sensitivity, tone cut-off, and carrier. The volume knob 402 is used to adjust the volume of the sound indicators from monitor circuit 109. The tone knob 404 adjusts the frequency of an audible tone that is used to communicate the level of conductivity between the probes to ensure proper probe contact with the patient's skin. The intensity knob 406 controls the carrier voltage. Sensitivity/baseline calibration knob 412 adjusts the conductivity of the patient treatment unit. The tone cut-off knob 410 adjusts the response level at which an auditory signal will be heard. The carrier knob 408 controls the frequency of the carrier wave. Initially, volume knob 402 is set to level of 5, and tone knob 404 is set to a level of 0. Additionally, intensity knob 406 is set to a level of 10, and the sensitivity/baseline calibration knob 412 is set to a level of 5 Likewise, the tone cutoff knob 410 is set to a level of 0, and the carrier knob 408 is set to a level of 10.

Returning to FIG. 6A, in block 601 the pain is characterized. For example, in conjunction with a patient history and examination results, the patient may characterize the location and severity of the pain. The patient may describe his or her pain in order to determine the scope and size of the problem and to establish a baseline measure of the perceived pain. The patient may point out the precise location of the most intense source of discomfort. For example, the patient may use a single finger to point at and touch the exact center of the pain point. Similarly, the physician may palpate the general area until the patient confirms the exact location of the most intense pain-related trigger point. The physician may continue to palpate the area to find a secondary trigger point. Once the location of the pain is identified and characterized, in block 605 the physician notes the displayed treatment time code prior to beginning the treatment as discussed above and shown in FIG. 4B.

Impedance readings are used to determine the condition of the tissue under examination. A reduction in impedance during or after treatment indicates the treatment is reducing the level of pain perceived by the patient. The patient treatment unit of the present disclosure makes real-time impedance readings during treatment. The impedance measurements may be monitored during treatment to determine the efficacy of the treatment. If the impedance measurements are lower than the initial measurement, the treatment is effective and continues until a minimum impedance measurement is observed, such as when additional treatment does not result in a lower impedance measurement or the impedance measurement begins to rise, even as additional treatment is applied. If the real-time impedance measurements continue to decline during treatment, treatment may be continued. The patient treatment unit of the present disclosure is configured to provide an on-going, real-time indication or display of the tissue impedance.

In block 607, the physician can select a narrow or diffuse mode of treatment using switch 318 shown in FIG. 3A. The physician then places the primary vibrating spherical probe 103 on the primary pain-related trigger point and the secondary vibrating spherical probe 105 on the secondary pain-related trigger point and notes the reading on the contact level display 214 as also shown in FIG. 2. The physician then adjusts the sensitivity knob 412 until a reading of 7 is achieved on the contact level display 214. The impedance indicator provides a relative reading that is set to illuminate one of the lower value LEDs. A change in impedance can be detected throughout the treatment. Because impedance may vary between people and between different anatomical parts of the same person (e.g., calloused areas of the foot versus stomach or inner arm), the relative change in impedance is primary indicator of the efficacy of the treatment.

In block 609, if a reading of 7 cannot be achieved initially, the contact between the patient's skin and the probes 103, 105 may be inadequate, and the physician can clean the patient's skin at the pain related trigger points in block 611. For example, the physician can clean the patient's skin with an isopropyl alcohol swab and return to block 607 to place the probes 103, 105 firmly into the patient's skin and, if a reading of 7 still cannot be achieved, the physician can move the probes to an adjacent point on the patient's skin where a contact display reading of 7 can be achieved.

Once a contact display reading of at least 7 is achieved, in block 611 the physician turns the probe intensity dial 344 fully toward the back of the vibrating spherical probe 103 indicative of minimum intensity. In block 613, the physician pushes the treatment switch 333 forward on the primary treatment probe as shown in FIG. 3C to select the treatment mode.

In block 615, the physician begins to adjust the intensity dial 344 forward on the primary treatment probe 103 as shown in FIG. 3C. As indicated above, the intensity of the electrical pulse train may be controlled by increasing or decreasing the pulse width, or by increasing or decreasing the carrier current. As the physician gradually increases the intensity of the electrical pulse train, the physician monitors the patient until the patient begins to feel the carrier current. The carrier current may feel like a tingly, or prickly sensation.

In block 617, the physician asks the patient to indicate when the intensity is strong and may reach the point of discomfort. Although higher intensity provides further pain relief, the patient should not experience significant discomfort. In block 619, the physician incrementally reduces the intensity of the pulse train, and returns to block 615 to optimize the intensity of the pulse train to just before the point where the intensity level has reached the maximum intensity level in which the patient remains comfortable. In block 621, the physician notes the impedance measurement of the area to be treated. The treatment then continues in block 623 for approximately 30 seconds as the physician monitors the impedance of the treated area in real-time. It is possible during treatment that the patient will begin to feel the treatment more strongly. If this occurs in block 625, the physician may use the intensity dial 344 on the primary vibrating spherical probe 103 to reduce the intensity of the electrical pulse train to a comfortable level.

After treating the affected tissue area for approximately 30 seconds, in block 627, the physician notes the impedance measurement of the treated area. Throughout the treatment process, the physician monitors and notes the impedance measurement of the treated area and continues treatment if the impedance measurements continue to drop. Also throughout the treatment process, the physician then asks the patient to reassess his or her pain.

If pain remains in block 631, and the impedance measurements continue to decline, the physician continues to treat the pain in block 633 and moves to the next set of pain trigger points and repeats blocks 615 to 631. If the pain is remediated, or if the pain is insignificant after treatment, or if further progress in ameliorating the pain may not be made based upon the monitored impedance measurements, the physician notes the impedance measurement in block 635, notes the treatment time code in block 637, and ends the treatment.

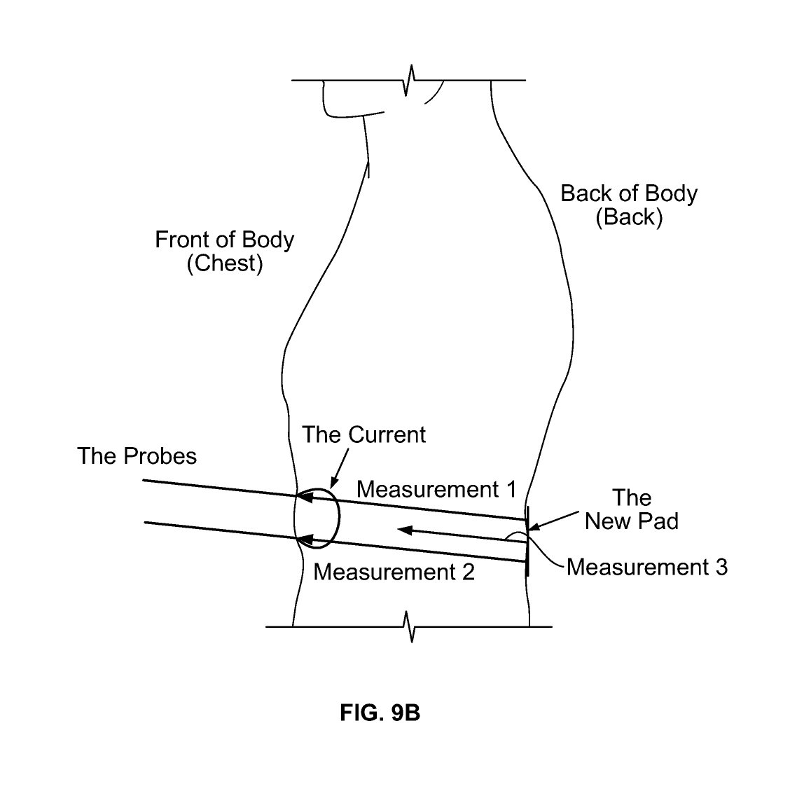

In many clinical environments, the electrical stimulation treatment is performed on the same side of the body. That is, normally, both probes are placed on the same side of the body. For example, to treat abdominal pain, both probes on the abdomen approximately 2-3 inches apart as shown in FIG. 9A (as reference numeral 901) instead of placing a probe on either side of the body and delivering current through the body. As shown in reference numeral 951 of FIG. 9A, a system and method of the present disclosure may utilize a large pad, for example 4″ by 4″ on the back of the patient as a third contact point while the primary and secondary probes are placed in contact with the patient's skin on their abdomen (as shown in 901).

As shown schematically in FIG. 9B, the additional 4″×4″ pad may be used to measure and record the distance from each individual probe tip to the pad, as well as to measure the closest distance of the current to the pad. This enables a user to measure the depth that the treatment current penetrates into the body by then subtracting the distance measured to the closest point detected of the current from the distance to the probe tips.

While the present inventions have been described in connection with a number of exemplary embodiments and implementations, the present inventions are not so limited, but rather cover various modifications, and equivalent arrangements, which fall within the purview of prospective claims.

What is claimed is:

1. A patient treatment unit for analyzing and treating pain in human or animal tissues, the treatment unit comprising:
a probe stimulus generator circuit that outputs a sequence of electrical pulses, the electrical pulses having a pulse width and a pulse frequency, the probe stimulus generator controlling the pulse frequency and the pulse width of the electrical pulses;
a primary vibrating spherical probe and a secondary spherical probe for contacting a body of a patient and electrically coupled to the probe stimulus generator to receive the sequence of electrical pulses;
a body impedance analysis circuit that senses voltage or current via the primary vibrating spherical probe and the secondary spherical probe in real-time as the sequence of electrical pulses are applied to the tissues when the probes are contacting the body of the patient; and
a monitor device electrically coupled to the body impedance analysis circuit that provides an indication of the sensed voltage or current as an impedance measurement in real-time as the sequence of electrical pulses are applied to the tissues.

2. The patient treatment unit of claim 1 further comprising:
a treatment counter circuit that detects and tracks an elapsed treatment time indicative of the time the primary vibrating spherical probe is receiving the sequence of electrical pulses.

3. The patient treatment unit of claim 2, wherein the treatment counter circuit is electrically coupled to the probe stimulus generator circuit and includes a handshake circuit that enables output of the sequence of electrical pulses from the probe stimulus generator when the treatment counter circuit is in a ready condition.

4. The patient treatment unit of claim 1, wherein the pulse width of the electrical pulses is in a range of substantially 0.24-0.74 milliseconds.

5. The patient treatment unit of claim 1, wherein the pulse frequency of the electrical pulses is in a range of substantially 1-490 Hz±6% with 500 ohms to 1 mega-ohm of resistance across the probes.

6. The patient treatment unit of claim 1, wherein the monitor device electrically coupled to the body impedance analysis circuit includes an audio output with a frequency cut off volume that provides an indication of the sensed voltage or current.

7. The patient treatment unit of claim 1 further comprising:
a coil sense circuit that evaluates the presence of a probe connection and enables the probe stimulus generator when the probes are connected to the body impedance analysis circuit.

8. The patient treatment unit of claim 1 further comprising:
a wall wart power supply circuit that provides a 12 volt DC power source to the patient treatment unit.

9. The patient treatment unit of claim 8, wherein the power supply circuit is electrically coupled to the probe stimulus generator circuit and includes a handshake circuit that enables output of the sequence of electrical pulses from the probe stimulus generator when the power supply circuit is in a ready condition.

10. The patient treatment unit of claim 1 further comprising:
a programming and debugging circuit that is used to configure the patient treatment unit and to debug processing errors in the patient treatment unit.

11. The patient treatment unit of claim 1, wherein the pulse frequency of the electrical pulses is in a range of substantially 4 kHz-20 kHz±6% with 500 ohms to 1 mega-ohm of resistance across the probes.

12. The patient treatment unit of claim 1 further comprising:
a response level circuit that is used to measure and indicate the conductivity between the probes.

13. The patient treatment unit of claim 1 further comprising:
an intensity adjustment circuit that is used to measure and indicate and adjust the intensity of the electrical pulses.

14. The patient treatment unit of claim 13, wherein the intensity of the electrical pulses is adjusted by adjusting a carrier current.

15. The patient treatment unit of claim 14, wherein the maximum current output of the probe stimulus generator circuit is 8.9 milliamps.

16. The patient treatment unit of claim 1, wherein the maximum charge of the electrical pulses is 7 micro-coulombs.

17. The patient treatment unit of claim 1, wherein the body impedance analysis circuit measures surface and tissue impedance of the patient in real-time using the sensed voltage or current from the probes.

18. The patient treatment unit of claim 17, wherein the sequence of electrical pulses output by the probe stimulus generator includes a stimulus profile that is based upon the surface and tissue impedance of the tissue of the patient.

19. The patient treatment unit of claim 18, wherein the stimulus profile is an inverse wave form of the impedance response of the tissue impedance of the tissue of the patient.

20. The patient treatment unit of claim 1, wherein the body impedance analysis circuit measures electrical phase of the voltage and current sensed from the probes.

21. The patient treatment unit of claim 1, wherein the body impedance analysis circuit includes a filtering circuit in which waveform ripples in the sensed voltage or current are corrected.

22. The patient treatment unit of claim 1 further comprising:
an isolated data logger circuit that measures and logs body impedance measurements without exposing the body to non-treatment currents.
